(19) United States
(12) Patent Application Publication
(10) Pub. No.: US 2019/0038306 A1
(43) Pub. Date: Feb. 7, 2019

(54) MINIMALLY INVASIVE INCISION INSTRUMENT HAVING A GUIDED CUTTING APPARATUS FOR MULTIPLE USE

(71) Applicant: Aesculap AG, Tuttlingen (DE)

(72) Inventors: STEPHAN LINDNER, Wurmlingen (DE); MATTHIAS WINKLER, Gomadingen (DE)

(21) Appl. No.: 16/075,016

(22) PCT Filed: Jan. 27, 2017
(86) PCT No.: PCT/EP2017/051722

§ 371 Date: Aug. 2, 2018

(30) Foreign Application Priority Data
Feb. 3, 2016 (DE) 10 2016 101 915.1

(51) Int. Cl. A61B 17/3211; A61B 17/32; A61B 17/3209

(52) U.S. Cl. CPC A61B 17/3211; A61B 17/3423; A61B 17/32093; A61B 17/32002

(57) ABSTRACT

An incision instrument for performing incisions in a standardized manner when opening an operation field includes a handle element with a cutting blade receiving element formed at a distal end of the handle element, a cutting blade which is removably receivable in the cutting blade receiving element as well as a guide channel that extends in the longitudinal direction of the instrument for receiving a guide wire therein.

### CROSS-REFERENCE TO RELATED APPLICATIONS

This application is the United States national phase entry of International Application No. PCT/EP2017/051722, filed Jan. 27, 2017, which is related to and claims the benefit of priority of German Application No. 10 2016 101 915.1, filed Feb. 3, 2016. The contents of International Application No. PCT/EP2017/051722 and German Application No. 10 2016 101 915.1 are incorporated by reference herein in their entireties.

### FIELD

The present invention relates to a minimally invasive surgical incision instrument for opening a surgical field, for example in the context of dorsal spinal stabilization.

### BACKGROUND

In surgical procedures, it is generally advantageous to minimize tissue injuries as much as possible, thus ensuring the best possible chances of recovery. With regard to opening up a surgical field, this means making tissue incisions as small as possible and with a clean cut. However, the tissue incisions should at best be made directly in such a size that the instrumentation required for the operation to be performed can be performed without subsequent incision enlargement. A subsequent incision enlargement always has the disadvantage of a time delay of the course of the operation as well as additional effort for the surgeon.

It is known to use a guide wire for positioning instruments, which is inserted into the body with a cannula and fixed in the required position. Such a guide wire, also referred to as a K-wire, can be attached to a vertebral body during spinal surgery, for example. After placement of the wire and removal of the cannula, the surgical field is opened with an incision instrument using the previously inserted wire as a positioning aid.

A surgical disposable incision instrument is known from the prior art for opening an operation field. It consists of a handle whose distal end is designed as a (one-piece) blade section. The handle and the blade section form a continuous channel in the longitudinal direction of the instrument to accommodate a guide wire. With this instrument, however, it is a disadvantage that the blades are not replaceable per se. As a result, it is realized as a very expensive disposable instrument, since regrinding the blades is generally not possible or only with difficulty due to the channel mouth in the blade area, among other things.

Furthermore, a so-called fascia separator is known from the prior art, in which a replaceable, one-piece disposable blade is attached to an instrument shaft and connected to it by means of screwing. For this purpose, the disposable blade has an internal thread and is held in a plastic sleeve. It also has a central, axially continuous opening extending in the direction of the instrument shaft, into which the guide wire is inserted during the operation before a fascia is severed, which is guided through the shaft of the instrument after passing through the blade. After the desired cutting of a fascia guided by means of the guide wire, the blade is removed from the operating area, released from the instrument shaft and discarded. However, the disadvantage of this prior art is that the blade is very expensive due to its elaborate design as described above. Another disadvantage is that the assembly of the blade on the instrument shaft and thus the preparation of the instrument for fascial separation is complex and time-consuming. Finally, the disadvantage is that the blade is only suitable for cutting through fascia due to its shape and not for cutting and separating other types of tissue such as skin, fatty or muscle tissue.

### SUMMARY

Against this background, the present invention is based on the object of providing an instrument, in particular an incision instrument, for a cost-effective standardized incision during opening an operation field, especially in the context of dorsal spinal stabilization and for percutaneous implantation of a pedicle screw system.

According to the present invention, this object is achieved by an instrument, in particular an incision instrument for standardized/predefined incision during opening an operation field, comprising a handle element with a cutting blade receiving element formed at a distal end of the handle element, at least one cutting blade which can be removably received or is removably received (preferably by clip connection) in or at the cutting blade receiving element, and a guide channel extending in the longitudinal direction of the instrument (i.e. extending along the cutting blade receiving element and/or the handle element) or a number of guide gates for receiving a (separate) guide wire/K-wire therein. It can also be said that, unlike the prior art, an incision instrument is provided which has at least one replaceable scalpel blade, in particular an instrument with replaceable single-use scalpel blades for opening the surgical field along a previously inserted and anchored K-wire. The invention therefore provides a particularly cost-effective instrument for a standardized incision when opening an operation field, especially in the context of percutaneous implantation of a pedicle screw system, since only the blade and not the entire instrument is intended as a single product. A particular advantage of the invention is that a conventional disposable scalpel blade can be used as the at least one cutting blade. The at least one cutting blade has at least one edge or cutting edge, preferably two cutting edges (facing away from each other), which preferably taper in the distal direction.

Complex cuts can be made quickly and easily by providing several cutting blades, for example two of them, which are held parallel or almost parallel to each other on/in the cutting blade receiving element. More specifically, two cutting blades are preferably provided, each with double-sided cutting edges, which are aligned to be parallel or substantially parallel or at an acute angle to each other, each tapering in the distal direction. In other words, this results in a shape of a double spearhead, so to speak, or in other words a double tip with two cutting edges facing away from each other and two cutting edges facing each other, the latter defining a cutting gap that converges pointedly in proximal direction.

The cutting blade receiving element can be designed very simply in the manner of a standard cutting blade receiving element. Preferably, the cutting blade receiving element has a flat side for applying the at least one, preferably the two cutting blade(s), as well as at least one, preferably two latching projections/latching devices for latching/clipping/pressing the cutting blade(s) on/in the cutting blade receiving element or the flat side thereof.

In particular, the following advantages can be achieved with the invention:

Significant cost savings through the use of replaceable and inexpensive scalpel blades,

sharp blades are always available for cutting through skin and muscle tissue,

scalpel blades with an inner and an outer cutting edge for severing the tissue with a defined cutting width up to the K-wire.

Advantageous embodiments of the invention are explained in more detail below.

In one embodiment, the guide channel may have a first guide channel opening distally to insert the guide wire and a second guide channel opening proximally for leading out the guide wire passing through the guide channel. Preferably, the first guide channel opening is formed at the level of an edge of the cutting blade(s) placed in the cutting blade receiving element. However, it can also be offset forward or backward with respect to the cutting edge. The guide channel is preferably formed on or in the handle element, for example is realized by a bore or similar cavity made in the handle element or by a hollow cylinder element arranged thereon.

It is particularly advantageous if the incision instrument has two cutting blade receiving elements positioned next to each other for one cutting blade to be arranged therein in each case. The instrument further has the two cutting blades mentioned above, each with at least one cutting edge, preferably two cutting edges. The guide channel may be arranged so as to be situated at least approximately in the middle and/or the guide channel opening may be centrally arranged between the two cutting blade receiving elements.

A particularly advantageous embodiment of the invention is characterized in that the incision instrument has four edges/cutting edges, namely the two inner cutting edges (facing each other) facing the guide channel and the two outer cutting edges facing away from the guide channel and oriented outwards. These can be formed by a single four-fold cutting blade or by two double-edged cutting blades. It is particularly preferred if the four blades or the four blades together with the guide channel tip form the shape of an M whose two upper tips form cutting tips of the instrument blade, which are first pierced into the tissue during an incision. In particular, the leading edge of the guide channel opening designed as a cannula tip may be positioned to be situated at the level of the two inner cutting edges and, together with these, form a substantially continuously extending cutting profile.

It is also advantageous if the distal end of the guide channel surrounding the guide channel opening is pointedly tapered like a cannula. It can also be said that in this case the tip of the guide channel can form an edge or part of the edge of the instrument, together with the other edges of the cutting blades placed in the cutting blade receiving element.

In one embodiment of the invention, the second guide channel opening is essentially located at the level of the proximal end of the cutting blade receiving element. Alternatively or additionally, the guide channel may be designed so as to extend substantially continuously through the handle element in the longitudinal direction and have an outlet opening at the proximal end or in a proximal end region of the handle element for a guide wire passing through the guide channel.

BRIEF DESCRIPTION OF THE DRAWING FIGURES

Further features and advantages of the present invention will be apparent from the following exemplary and non-restrictive description of the invention with the aid of Figures. These are only schematic in nature and serve only to understand the invention. In the Figures:

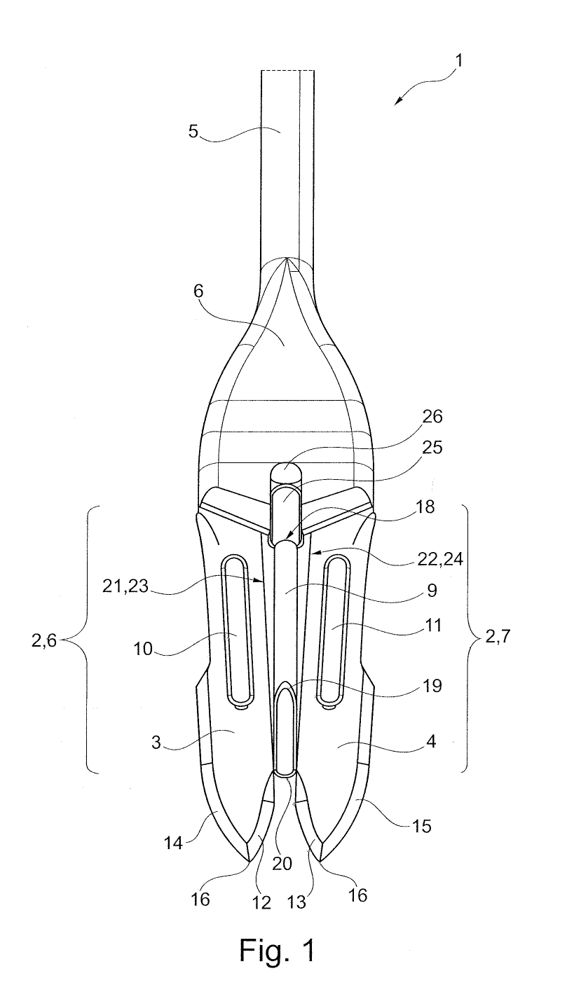

FIG. 1 is a schematic plan view of a distal section of an incision instrument according to the invention,

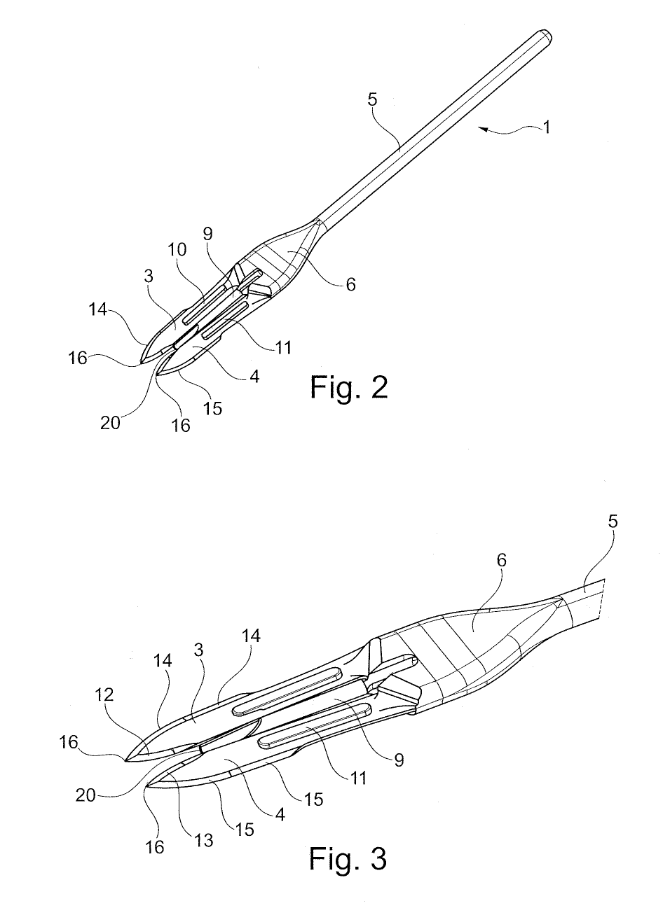

FIG. 2 is a schematic perspective view of the entire incision instrument according to the invention, in particular of the instrument according to FIG. 1,

FIG. 3 is a schematic perspective view of a distal section of the incision instrument of FIG. 2,

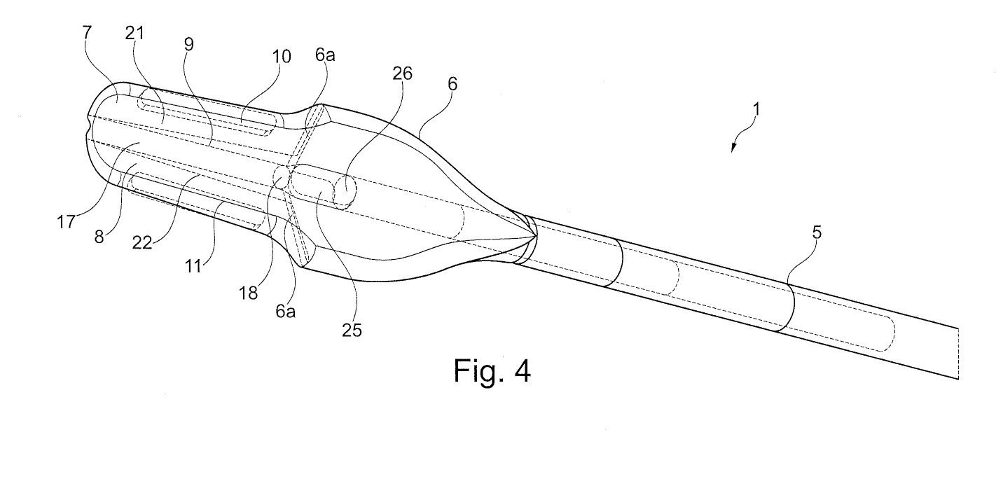

FIG. 4 is a front perspective view of a mounting head of the incision instrument according to the invention without cutting blades mounted thereon,

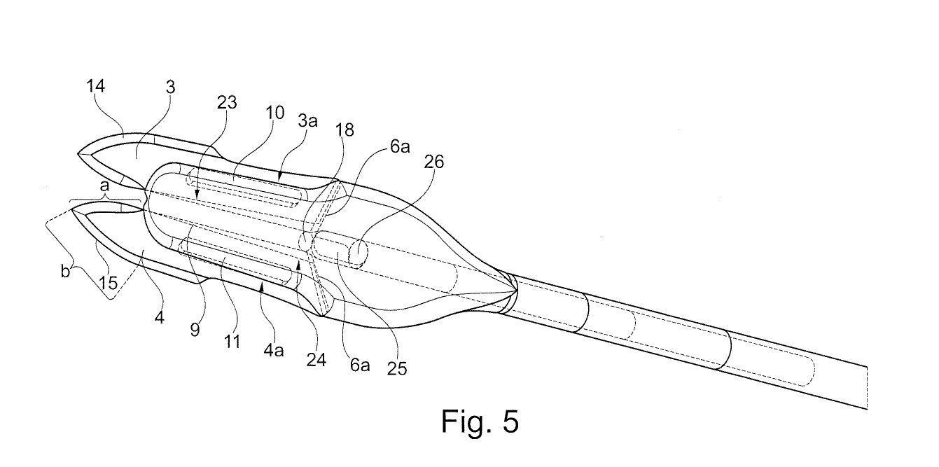

FIG. 5 is a front perspective view of a mounting head of the incision instrument according to the invention with cutting blades mounted thereon, whereby these are shown transparent for clarification of the assembly engagement, and

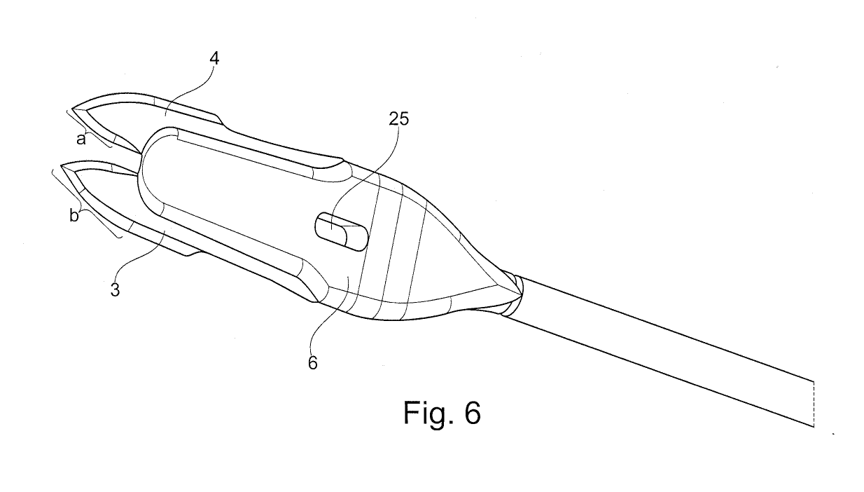

FIG. 6 is a rear view of a mounting head of the incision instrument according to the invention with cutting blades mounted thereon.

### DETAILED DESCRIPTION

FIG. 1 shows as an example of the invention an incision instrument 1 with two cutting blades 3, 4 mounted in/on its cutting blade receiving element 2. The instrument 1 shown serves to open up an operation field (e.g. skin incision) not shown along a guide wire, also not shown in the Figures, for example in the form of a K-wire, which was fixed to a patient bone at certain points beforehand by default.

The incision instrument 1 has a proximal instrument handle or handle element 5 and is preferably made of stainless steel customary for surgical instruments or any other suitable material. The distal end section of the handle element 5 is designed as a mounting head 6 which widens in relation to the handle element 5 and is flattened at least on one (front) side. A first cutting blade receiving element 7 and a second cutting blade receiving element 8 are formed in a distal flat section of the mounting head 6, as described in more detail below. The two cutting blade receiving elements 7, 8 extend in the longitudinal direction of the instrument and are spaced apart in its width direction. Preferably, the two cutting blade receiving elements are arranged in the same plane with respect to the central axis of the instrument in such a way that cutting blades mounted on it define a common plane. Alternatively, the two cutting blade receiving elements 7, 8 can also be set at an (obtuse) angle (not equal to 0°) to each other.

A guide channel 9 for a guide wire (K-wire) not shown is formed or arranged between the two cutting blade receiving elements (support surfaces/planes) 7, 8 on the handle element 5 or the mounting head 6, which therefore preferably also involves the symmetry line of the mounting head 6.

The two cutting blade receiving elements 7, 8 each hold one of the cutting blades 3, 4. These are preferably commercial disposable scalpel blades, and the cutting blade receiving element 7, 8 is designed in a suitable, usual way. A clamping projection 10, 11, which engages through a groove 3a, 4a formed for this purpose in the corresponding cutting blade 3, 4, can be seen in the Figures for each cutting blade receiving element 7, 8, whereby a clamping or latching connection is formed between the clamping projection 10, 11 and the groove 3a, 4a and the cutting blade 3, 4 is held in exact position on the cutting blade receiving element 7, 8. If the cutting blade 3, 4 is positioned as intended, a stop or a stop edge 6a is formed between the mounting head 6 and the end of the cutting blade 3, 4 facing it, as an additional (axially acting) support and positioning aid, as can be seen in particular from FIG. 5. FIGS. 4 to 6 clearly show that the mounting head 6 is designed with flattened or rounded edges, which ensures particularly good cleanability and a low risk of unintentional tissue damage in the vicinity of the incision site.

The cutting blades 3, 4 are double-sided cutting blades (blade with two cutting edges) or commercial scalpel blades with one inner cutting edge 12 and 13 directed towards the guide channel 9 and one outer cutting edge 14 and 15 facing away from the guide channel 9. The inner cutting edges 12 and 13 and the outer cutting edges 14 and 15 meet at a cutting tip 16 in each case. This creates a cutting tool with two cutting blades 3, 4 spaced apart in the width direction of the mounting head 6 and thus two instrument tips.

The inner cutting edges 12, 13 of each cutting blade 3, 4 extend over a length a from the cutting edge tip 16 backwards, i.e. in the proximal direction relative to the mounting head 6. The external cutting edges 14, 15 extend over a length b from the cutting edge tip 16 backwards. In the instrument 1 shown, length a is shorter than length b, so that the outer cutting edges 14, 15 extend further towards the mounting head 6 than the inner cutting edges 12, 13, resulting in a (cutting) gap distally to the guide channel between the two opposite inner cutting edges 12, 13, which narrows proximally (essentially) conically.

The guide channel 9, which adjoins the cutting gap proximally, is formed in this case by a tubular section of handle element 5 with an insertion opening 17 (see FIG. 5) at the distal end and an outlet opening 18 at the proximal end, said tubular section extending between the cutting blade receiving elements 7, 8 in the longitudinal direction of the instrument 1/mounting head 6. The guide channel 9 extends continuously through the entire tubular section, which is preferably formed in one piece with the mounting head 6. At its distal end, i.e. the end forming the insertion opening 17, the tubular section or in other words the wall 19 surrounding the guide channel 9 is chamfered/tipped in the manner of a cannula and thus formed with a channel tip/channel front edge 20.

The cutting blades 3, 4 are arranged on both sides of the guide channel 9 and arranged in stable position on the mounting head 6 in such a way that the channel tip/channel front edge 20 of the guide channel 9 and the two inner cutting edges 12, 13 form an (essentially) continuous, curved or angled cutting profile. In summary, it can also be said that the incision instrument 1 in distal direction has an approximately M-shaped cutting edge serially composed of the outer cutting edge 14, the cutting tip 16 and inner cutting edge 12 of the first cutting blade 3, channel tip/channel front edge 20 and finally the inner cutting edge 13, cutting tip 16 and outer cutting edge 15 of the second cutting blade 4 (in this order).

According to the invention, between the guide channel 9 and the cutting blade receiving elements 7, 8, an abutment shoulder 21 or 22 may basically be formed in the longitudinal direction of the instrument, which is indicated particularly in FIG. 4 (without cutting blade); on said abutment shoulders, respectively one cutting blade 3, 4 rests and is supported with its abutment edge 23 or 24 adjoining the inner cutting edge 12 or 13 on the mounting head side, as shown in particular in FIG. 5.

In the area of the proximal end of the guide channel 9, i.e. in the area immediately adjoining its outlet opening 18, the mounting head 6 has a central (oval or rectangular) recess 25 (preferably slot-like opening extending perpendicular to the cutting blade receiving element/surfaces), which thus connects the front and rear sides of the mounting head 6. The distal end of recess 25 is adjoined by a kind of outlet opening (possibly with a longitudinally extending concave/trough-shaped guide surface) 26 of a guide channel formed in the (shaft-like) instrument handle 5, which is intended to facilitate leading out the guide wire from the outlet opening 18 and the recess 25 as well as inserting it into the guide channel in the instrument handle.

However, it is also possible not to thread the guide wire into the guide channel in the instrument handle 5, but to lead it out of the recess 25 (at the front or back) in the area of the mounting head 6 and then hold it parallel to the instrument handle 5-on the outside.

1.14. (canceled)

15. An incision instrument adapted for predefined incision guidance when opening an operation field, the incision instrument comprising:
a handle element comprising a cutting blade receiving element formed at a distal end of the handle element and preferably designed as a reusable component,
at least one cutting blade which is removably accommodated in the cutting blade receiving element and preferably designed as a single-use component, and
a guide channel extending in the longitudinal direction of the instrument for receiving a guide wire therein, wherein the incision instrument has two cutting blade receiving elements positioned side by side for in each case one cutting blade to be arranged therein or thereon, and the two cutting blades each form a blade tip and are aligned so as to converge to each other at an acute angle towards distal.

16. The incision instrument according to claim 15, wherein the guide channel has distally a first guide channel opening for inserting the guide wire and proximally a second guide channel opening for leading out the guide wire passing through the guide channel.

17. The incision instrument according to claim 15, wherein the first guide channel opening is formed at the level of an edge of the cutting blade placed in the cutting blade receiving element.

18. The incision instrument according to claim 16, wherein a distal end of the guide channel surrounding the first guide channel opening is of a cannula-like spray-cast construction.

19. The incision instrument according to claim 16, wherein the guide channel is arranged so as to be situated at least approximately in the middle and/or the first guide channel opening is arranged centrally between the two cutting blade receiving elements.

20. The incision instrument according to claim 16, wherein the second guide channel opening is arranged substantially at the level of the proximal end of the cutting blade receiving element.

21. The incision instrument according to claim 15, wherein the guide channel is constructed such that it extends through the handle element completely in its longitudinal direction and has an outlet opening at the proximal end or in a proximal end region of the handle element for a guide wire passing through the guide channel

22. The incision instrument according to claim 16, wherein the front edge of the first guide channel opening, which is designed as a cannula tip, is positioned at the level of two inner cutting edges and together with these forms a substantially continuously extending cutting edge profile.

23. The incision instrument according to claim 15, wherein the cutting blade is a disposable scalpel blade and the cutting blade receiving element is designed with a latching or clipping device for each cutting blade.

24. The incision instrument according to claim 15, wherein the guide channel is arranged or formed on or in the handle element.

25. An incision instrument adapted for predefined incision guidance when opening an operation field, the incision instrument comprising:
a handle element comprising a cutting blade receiving element formed at a distal end of the handle element and designed as a reusable component,
at least one cutting blade which is removably accommodated in the cutting blade receiving element and designed as a single-use component, and
a guide channel extending in the longitudinal direction of the instrument for receiving a guide wire therein, wherein the incision instrument has four edges or cutting edges, two inner cutting edges facing the guide channel and two external cutting edges facing away from the guide channel and oriented towards outside.

26. The incision instrument according to claim 25, wherein the two cutting blades each form a blade tip and are aligned parallel to one another or so as to converge to each other at an acute angle towards distal and wherein each of the two cutting blades has two of the four cutting edges, the two inner cutting edges defining a cutting gap narrowing in proximal direction.

27. The incision instrument according to claim 25, wherein the guide channel has distally a first guide channel opening for inserting the guide wire and proximally a second guide channel opening for leading out the guide wire passing through the guide channel.

28. The incision instrument according to claim 25, wherein the first guide channel opening is formed at the level of an edge of the cutting blade placed in the cutting blade receiving element.

29. The incision instrument according to claim 27, wherein a distal end of the guide channel surrounding the first guide channel opening is of a cannula-like spray-cast construction.

30. The incision instrument according to claim 27, wherein the guide channel is arranged so as to be situated at least approximately in the middle and/or the first guide channel opening is arranged centrally between the two cutting blade receiving elements.

31. The incision instrument according to claim 27, wherein the second guide channel opening is arranged substantially at the level of the proximal end of the cutting blade receiving element.

32. The incision instrument according to claim 25, wherein the guide channel is constructed such that it extends through the handle element completely in its longitudinal direction and has an outlet opening at the proximal end or in a proximal end region of the handle element for a guide wire passing through the guide channel.

33. The incision instrument according to claim 27, wherein the front edge of the first guide channel opening, which is designed as a cannula tip, is positioned at the level of two inner cutting edges and together with these forms a substantially continuously extending cutting edge profile.

34. The incision instrument according to claim 25, wherein the cutting blade is a disposable scalpel blade and the cutting blade receiving element is designed with a latching or clipping device for each cutting blade.

35. The incision instrument according to claim 25, wherein the guide channel is arranged or formed on or in the handle element.
